(19) United States
(12) Patent Application Publication
(10) Pub. No.: US 2016/0176813 A1
(43) Pub. Date: Jun. 23, 2016

(54) PROCESS FOR THE OXIDATION OF CYCLOHEXANE

(71) Applicant: INVISTA NORTH AMERICA S.A. R.L., Wilimington, DE (US)

(72) Inventor: David Lee VALDEZ, Victoria, TX (US)

(73) Assignee: INVISTA NORTH AMERICA S.A R.L., Wilmington, DE (US)

(21) Appl. No.: 14/415,176

(22) PCT Filed: Jul. 19, 2012
(86) PCT No.: PCT/US12/47400

§ 371 Date: Feb. 15, 2016

(51) Int. Cl. C07C 407/00; C07C 29/50; C07C 45/33

(52) U.S. Cl. CPC C07C 407/00; C07C 45/33; C07C 29/50; C07C 2101/14

(57) ABSTRACT

A reaction zone is provided comprising a plurality of oxygen clean up zones and a plurality of oxidation zones. A first stream is introduced to oxidation clean up zones comprising liquid cyclohexane, and optionally a cyclohexane oxidation catalyst. In addition, an oxygen containing gas is introduced into the oxidation zones. Next, the first stream is passed downwardly from the oxygen clean up zones to the oxidation zones, while cross-currently passing the oxygen containing gas upwardly from the oxidation zones to the oxygen clean up zones. The oxygen containing gas is introduced directly into the first stream as it travels through the oxidation zones, and the reaction between the first stream and the oxygen containing gas produces a product mixture. Finally, a product mixture is withdrawn from the oxidation zones that comprises CHHP, cyclohexanone and cyclohexanol.

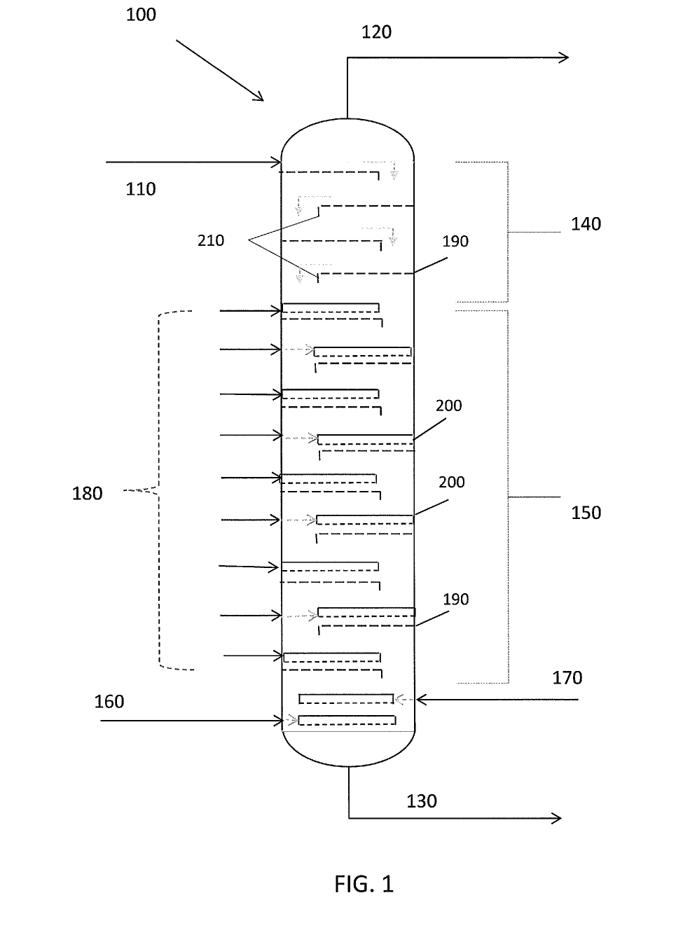

### FIELD OF THE INVENTION

This disclosure relates to a process for the air oxidation of cyclohexane. More specifically, it relates to an improved method for introducing an oxygen containing gas into a reaction zone containing liquid cyclohexane.

### BACKGROUND OF THE INVENTION

The air oxidation of cyclohexane is an important process for the production of caprolactam and adipic acid, which are used in the manufacture of synthetic products, such as nylon. The oxidation of cyclohexane by air produces a reaction product comprising cyclohexanol (A), cyclohexanone (K) cyclohexylhydroperoxide (CHHP) and small amounts of by-products. Cyclohexanone (K) and cyclohexanol (A) are the main product of the overall process and the mixture is commonly known as KA oil. Several patents, herein incorporated by reference, such as U.S. Pat. Nos. 3,530,185, 3,987,100, 5,780,683, 6,888,034 and 6,703,529 teach the preparation of a mixture containing cyclohexanol, cyclohexanone and cyclohexylhydroperoxide by the air oxidation of cyclohexane.

It is well known that CHHP in a mixture containing cyclohexanol, cyclohexanone, other products of the air oxidation reactions will react to form KA oil. However, this process does not result in a high yield of KA oil and other waste materials are formed. It has been found that the highest yields of KA oil can be achieved when the oxidation of cylcohexane is performed under conditions that result in a greater amount of CHHP. The CHHP is then treated by hydrogenation in a separate process to cyclohexanone (K) and cyclohexanol (A) to give an increased overall yield of KA oil. For example, the preparation of cyclohexanol and cyclohexanone from cyclohexylhydroperoxide by hydrogenation has been in described in U.S. Pat. Nos. 3,694,511 and 3,927,108, herein incorporated by reference.

The air oxidation reaction is generally conducted in reactors with multiple reaction stages. U.S. Pat. No. 6,075,169, herein incorporated by reference, teaches a method of cyclohexane oxidation oxygen wherein the gases are brought into contact with the liquid cyclohexane in a reaction zone. To achieve direct contact of the oxygen containing gas with the liquid cyclohexane, the reactor is designed to have no continuous vapor space. This configuration may result in back mixing of the oxidation products through the reaction zones and over oxidation of the desired oxidation products and a reduction in yield.

U.S. Pat. No. 3,957,876, herein incorporated by reference, teaches a method for cyclohexane oxidation in order to produce cyclohexanol (A), cyclohexanone (K) and CHHP. In this process, the cyclohexane oxidation process is carried out in several reaction zones with a continuous vapor space between zones. The oxygen containing gas is then added into the vapor space between each reaction zone and is distributed into the liquid in the zone above via a sieve tray. The addition of the oxygen containing gas in to the vapor space above the reaction zones limits the liquid residence time for the oxidation reaction. In addition, optimal mixing of the cyclohexane and oxygen containing gas is not achieved in this configuration.

Therefore, there is a need for an improved process for the air oxidation of cyclohexane, wherein the reaction zones are configured to achieve uniform distribution and maximum liquid residence time of the oxygen containing gas and prevent back mixing of the oxidation products between the reaction zones.

### SUMMARY OF THE INVENTION

The present invention relates to a process for the oxidation of cyclohexane wherein an oxidation containing gas is introduced into series of reaction zones. The oxygen containing gas is introduced directly into the liquid mixture within each reaction zone. The reaction zones are also configured to have a continuous vapor space to prevent back mixing of the oxidation products between reaction zones.

An embodiment of the present invention comprises the steps of;

a) providing a reaction zone comprising a plurality of oxygen clean up zones and a plurality of oxidation zones, wherein the oxygen clean up zones and the oxidation zones are in fluid communication, and wherein the reaction zone is configured so that the oxygen clean up zones and oxidations zones are contained in a single continuous vapor space;

b) introducing a first stream comprising liquid cyclohexane into the oxygen cleanup zones;

c) introducing an oxygen containing gas into the oxidation zones;

d) passing the first stream downwardly from the oxygen clean up zones to the oxidation zones, while cross-currently passing the oxygen containing gas upwardly from the oxidation zones to the oxygen clean up zones, wherein the oxygen containing gas is introduced directly into the first stream as it travels through the oxidation zones, and wherein the reaction between the first stream and the oxygen containing gas produces a product mixture; and

e) withdrawing a product mixture from the oxidation zones that comprises cyclohexylhydroperoxide (CHHP), cyclohexanone and cyclohexanol.

In another embodiment, the oxygen containing gas is introduced directly into the first stream at multiple oxidation zones.

In another embodiment, the oxygen containing gas is introduced uniformly over the cross section of each oxidation zone.

In another embodiment, the oxygen containing gas is introduced directly into the first stream at each individual oxidation zone through a gas conduit.

In another embodiment, the gas velocity of the oxygen containing gas exiting the gas conduit is in the range from about 5 m/s to about 50 m/s.

In another embodiment, the oxygen containing gas exits the gas conduit at an angle of about 10 to about 50 degrees from the vertical.

In another embodiment, the gas conduit comprises a gas sparger.

In another embodiment, the gas conduit comprises a pipe with a plurality of perforations.

In another embodiment, the perforations have a diameter in the range of about 2 mm to about 4 mm.

In another embodiment, the perforations point downward at an angle of about 10 degrees to about 50 degrees from the vertical.

In another embodiment, a portion of the perforations point downward at an angle to the right of the vertical and a portion of the perforations point downward at an angle to the left of the vertical.

In another embodiment, the gas velocity of the oxygen containing gas exiting the perforations is in the range from about 5 m/s to about 50 m/s.

In another embodiment, the oxidation zones are maintained at a temperature range of about 145° C. to about 170° C.

In another embodiment, the reaction between the first stream and the oxygen containing gas takes place in the presence of a cyclohexane oxidation catalyst.

In another embodiment, the cyclohexane catalyst comprises soluble salts of at least one metal selected from the group comprising of cobalt and chromium.

In another embodiment, the cyclohexane catalyst is a soluble cobalt salt selected from a group comprising cobalt naphthenate, cobalt octoate, cobalt laurate, cobalt palminate, cobalt stearate, cobalt linoleate, cobalt acetylacetonate and mixtures thereof.

In another embodiment, the reaction zone comprises a single reaction vessel.

### BRIEF DESCRIPTION OF THE DRAWING

The FIGURE is a process diagram for an embodiment of the present invention.

### DETAILED DESCRIPTION OF THE INVENTION

The present invention relates to a process for the oxidation of cyclohexane wherein an oxidation containing gas is introduced into series of reaction zones. The oxygen containing gas is introduced directly into the liquid mixture within each reaction zone. The reaction zones are also configured to have a continuous vapor space to prevent back mixing of the oxidation products between reaction zones.

All patents, patent applications, test procedures, priority documents, articles, publications, manuals, and other documents cited herein are fully incorporated by reference to the extent such disclosure is not inconsistent with this invention and for all jurisdictions in which such incorporation is permitted.

Referring now to the figure, an exemplary embodiment of the present invention is will now be described. The cyclohexane oxidation reactor 100 comprises oxygen clean up zones 140 and oxidation zones 150, which are in fluid communication. The oxidation zones 150 may consist of a plurality of trays 190 where the oxidation of cyclohexane can take place. The oxygen clean up zones 140 may consist of a plurality of trays 190 where the heat from the oxidation reaction can be recovered. The oxidation zones and oxygen clean up zones may be contained in multiple vessels. In an exemplary embodiment of the current invention, the oxygen clean up zones 140 and the oxidation zones 150 are contained in a single reactor vessel 100.

The liquid cyclohexane in stream 110 may contain fresh cyclohexane and/or liquid cyclohexane recycled from any subsequent part of the process. Stream 110 is added at the top of the reactor 100 and travels cross-flow across the trays 190 in oxidation clean up zones 140 and oxidation zones 150. A stream of cyclohexane oxidation catalyst (not shown) can optionally be added into the oxidation clean up zones. The catalyst may be any suitable cyclohexane catalyst known in the art, including soluble salts of cobalt or chromium, and mixtures thereof. If a cobalt catalyst is chosen, it may be selected from a group comprising cobalt naphthenate, cobalt octoate, cobalt laurate, cobalt palminate, cobalt stearate, cobalt linoleate, cobalt acetylacetonate and mixtures thereof.

The cyclohexane oxidation may also be carried out in the presence of a phosphoric ester, such as di(2-ethylhexyl)phosphoric acid. Such a process is disclosed in Lyon et al., U.S. Pat. No. 4,675,450, which disclosure is hereby incorporated by reference in the present application.

An oxygen containing gas is fed into the oxidation zones 150 at multiple points 180 and stream 160. Liquid cyclohexane stream 110 is fed downwardly from the oxygen clean up zones 140 to the oxidation zones 150, while cross-currently passing the oxygen containing gas upwardly from the oxidation zones 150 to the oxygen clean up zones 140, wherein the reaction between the first stream and the oxygen containing gas produces a product mixture 130. The product mixture 130 comprises cyclohexylhydroperoxide (CHHP), cyclohexanone and cyclohexanol and other oxidation products.

The cyclohexane oxidation reactor 100 is separated into multiple oxidation zones 150 and oxidation clean up zones 140 by sieve trays 190 fitted with downcomers 210. The trays 190 are spaced so there is a continuous vapor space within the reactor 100 during operation. During operation, each oxidation zone 150 and oxidation clean up zone 140 will contain a liquid phase and a vapor phase. Liquid cyclohexane 110 is added at the top of the reactor 100 and travels cross-flow across each oxidation clean up zone and oxidation zone in series entering and leaving via the downcomers 210. The downcomers 210 are sized so that gas-liquid separation occurs close to the top of the vapor phase between each sieve tray 190. Any separation means well known in the art may be used to create the oxidation zones 150 and oxidation clean up zones 140 within the reactor 100.

The oxygen-containing gas 160,180 is introduced into the oxidation zones 150 and reactor bottom by means of gas spargers 200. The gas spargers 200 are located above the sieve trays 190 within each oxidation zone 150. The gas spargers 200 are comprised of pipes fitted with perforations to distribute the oxygen-containing gas 160,180 within the liquid phase of each oxidation zone 150. The oxidation zones 150 are configured so that the gas spargers 200 distribute the oxygen containing gas uniformly over the cross section of each sieve tray 190 within each oxidation zone 150.

The perforations in the gas spargers 200 are designed to distribute the oxygen containing gas at a velocity in the range of about 10 m/s to about 50 m/s. In an exemplary embodiment of the invention, an average gas velocity of 25 m/s is maintained. The perforations are circular and have openings with diameters in the range of about 2 mm to about 4 mm. In other embodiments of the current invention, the perforations may be designed to have openings of any geographical shape. The geometric dimensions and number of perforations are calculated to result in the indicated gas velocity range. The perforations point downward within the liquid phase of the oxidation zones 150 and distribute the oxygen containing gas at an angle of about 10 to about 50 degrees from the vertical.

In an exemplary embodiment of the invention, the perforations are at 30 degrees from the downward vertical. They are arranged in pairs with one at 30 degrees to the left and one at 30 degrees to the right. It is understood that many configurations of the perforations may be used to distribute the oxygen containing gas 180 within the liquid phase of the oxidation zones 150. The flow of oxygen containing gas 180 in each gas sparger 200 is maintained high enough to prevent flow of liquid or gas from the oxidation zones 150 into the gas sparger. In addition, the pressure within each gas sparger 200 is kept sufficiently higher than the pressure within the oxidation zones 150 and is maintained such that an even gas flow through all of the perforations is achieved.

The oxygen containing gas 180 flows from the base of each oxidation zone 150 up and into the next oxidation zone or oxygen clean up zone 140 through the sieve trays 190. The gas exiting each oxidation zone 150 is depleted in oxygen. Additional oxygen containing gas 180 is added into each oxidation zone 150 to maintain an excess of unreacted oxygen in that oxidation zone. Inert gas 170 is also added via a similar gas sparger 200 below the oxidation zones 150.

The oxygen clean up zones 140 provides additional reaction volume to enable control of the unreacted oxygen in the gas 120 leaving the reactor 100. Vapor travels up from zone to zone via the spaces in the sieve trays 190. Liquid does not pass down through the tray spaces, but rather via the downcomers 210, as described above. The sieve trays 190 and downcomers 210 are designed to assure that the oxidation zones 150 and oxygen clean up zones are primarily filled with the vapor dispersed in the liquid phase with only a small vapor phase below each tray.

Above the oxygen clean up zones 140, the reactor may also contain a heat-transfer zone that condenses cyclohexane vapor from the off-gas 120 exiting the oxygen clean up zones 140 by direct contact with part of the liquid cyclohexane feed 110. This process preheats the feed 110 before it enters oxygen clean up zones 140.

In an exemplary embodiment of the current invention, the temperatures in the oxidation zones are maintained at a range of about 145° C. to about 170° C. The temperature of the product mixture 130 exiting the oxidation zones 150 is maintained a temperature in the range from about 145° C. to about 170° C.

Any unreacted oxygen (along with the nitrogen present in the air) leaves the reactor 100 as an off-gas stream 120. The off-gas 120 also contains vaporized cyclohexane and other compounds. The amount of unreacted oxygen in the off-gas is commonly referred to as “oxygen leakage.” The vaporized cyclohexane and other products in the off-gas are condensed and recovered, and the off-gases leave the system, usually to an abatement system. The oxidation products that are produced from the oxidation reaction are recovered from the liquid effluent from the reactor or reactors, and the unreacted cyclohexane is recycled.

To maintain safe levels of oxygen leakage, the temperature of the off-gas 120 exiting the oxygen clean up zones 140 is maintained a temperature in the range from about 110° C. to about 150° C. In an exemplary embodiment of the current invention, the oxygen leakage in the off gas is maintained at less than 3.0% by volume of unreacted oxygen, measured on a VOC-free basis. More preferably, oxygen leakage is maintained in a range from about 1.0% to about 2.0% by volume, measured on a VOC-free basis.

### EXAMPLES

The following Examples demonstrate the present invention and its capability for use. The invention is capable of other and different embodiments, and its several details are capable of modifications in various apparent respects, without departing from the scope and spirit of the present invention. Accordingly, the Examples are to be regarded as illustrative in nature and non-limiting.

### Example 1

The following example illustrates the method of the current invention as used at I NVISTA's Victoria cyclohexane oxidation process. The reaction zone in the Victoria plant contains 17 trays, which include the oxidation zones (base and trays 1-13) and oxidation clean up zones (trays 14-17). The temperatures of the materials in the column are measured on trays 2, 5, 8, 11, 14, and 17, as well as in the column base, tails line, and off-gas. Using the air flow distribution in Table 1, the temperature in the oxidation zones (base, and trays 1-13) has been observed between 145° C. and 170° C. The temperature in the base section (tails line) is 145-170° C. The temperature in the reactor off-gas temperature normally operates between 110-150° C. Air was injected into the oxidation zones (base and trays 1-13) through gas spargers having perforations with an average diameter of 3.2 mm. The perforations were positioned so that the air was injected into the oxidation zones downwardly at an angle 30° from vertical. The average gas velocity exiting the spargers was maintained between about 6.5 m/s to about 19 m/s. The cyclohexane conversion was in the range of 3.0% to 4.5% and the ratio of CHHP to cyclohexanone (K), cyclohexanol (A) and CHHP in the product was 0.50 to 0.65.

### Example 2

The following example is a method for the oxidation of cyclohexane. A reaction zone is provided comprising a plurality of oxygen clean up zones and a plurality of oxidation zones. The oxygen clean up zones and the oxidation zones are in fluid communication and are configured so there is a continuous vapor space throughout the reaction zone. A first stream is introduced to oxidation clean up zones comprising liquid cyclohexane, and optionally a cyclohexane oxidation catalyst. In addition, an oxygen containing gas is introduced into the oxidation zones. Next, the first stream is passed downwardly from the oxygen clean up zones to the oxidation zones, while cross-currently passing the oxygen containing gas upwardly from the oxidation zones to the oxygen clean up zones. The oxygen containing gas is introduced directly into the first stream as it travels through the oxidation zones, and the reaction between the first stream and the oxygen containing gas produces a product mixture. Finally, a product mixture is withdrawn from the oxidation zones that comprises CHHP, cyclohexanone and cyclohexanol.

### Example 3

The process of Example 2 is repeated with additional steps. In this example, the oxygen containing gas is introduced directly into the first stream at multiple oxidation zones.

### Example 4

The process of Example 3 is repeated with additional steps. In this example, the oxygen containing gas is introduced uniformly over the cross section of each oxidation zone.

### Example 5

The process of Example 4 is repeated with additional steps. In this example, the oxygen containing gas is introduced directly into the first stream at each individual oxidation zone through a gas conduit.

### Example 6

The process of Example 5 is repeated with additional steps. In this example, the gas velocity of the oxygen containing gas exiting the gas conduit is in the range from about 5 m/s to about 50 m/s.

### Example 7

The process of Example 6 is repeated with additional steps. In this example, the oxygen containing gas exits the gas conduit at an angle of about 10 to about 50 degrees from the vertical.

### Example 8

The process of Example 7 is repeated with additional steps. In this example, the gas conduit comprises a gas sparger.

### Example 9

The process of Example 8 is repeated with additional steps. In this example, the gas conduit comprises a pipe with a plurality of perforations.

### Example 10

The process of Example 9 is repeated with additional steps. In this example, the perforations have a diameter in the range of about 2 mm to about 4 mm.

### Example 11

The process of Example 10 is repeated with additional steps. In this example, the perforations point downward at an angle of about 10 degrees to about 50 degrees from the vertical.

### Example 12

The process of Example 11 is repeated with additional steps. In this example, a portion of the perforations point downward at an angle to the right of the vertical and a portion of the perforations point downward at an angle to the left of the vertical.

### Example 13

The process of Example 12 is repeated with additional steps. In this example, the gas velocity of the oxygen containing gas exiting the perforations is in the range from about 5 m/s to about 50 m/s.

### Example 14

The process of Example 13 is repeated with additional steps. In this example, the oxidation zones are maintained at a temperature range of about 145° C. to about 170° C.

### Example 15

The process of Example 14 is repeated with additional steps. In this example, the reaction between the first stream and the oxygen containing gas takes place in the presence of a cyclohexane oxidation catalyst.

### Example 16

The process of Example 15 is repeated with additional steps. In this example, the cyclohexane catalyst comprises soluble salts of at least one metal selected from the group comprising of cobalt and chromium.

### Example 17

The process of Example 16 is repeated with additional steps. In this example, the cyclohexane catalyst is a soluble cobalt salt selected from a group comprising cobalt naphthenate, cobalt octoate, cobalt laurate, cobalt palminate, cobalt stearate, cobalt linoleate, cobalt acetylacetonate and mixtures thereof.

### Example 18

The process of Example 17 is repeated with additional steps. In this example, the reaction zone comprises a single reaction vessel.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also the individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±8%, or ±10%, of the numerical value(s) being modified. In addition, the phrase “about ‘x’ to ‘y’” includes “about ‘x’ to about ‘y’”.

While the illustrative embodiments of the invention have been described with particularity, it will be understood that the invention is capable of other and different embodiments and that various other modifications will be apparent to and may be readily made by those skilled in the art without departing from the spirit and scope of the invention. Accordingly, it is not intended that the scope of the claims hereof be limited to the examples and descriptions set forth herein but rather that the claims be construed as encompassing all the features of patentable novelty which reside in the present disclosure, including all features which would be treated as equivalents thereof by those skilled in the art to which the invention pertains.

What is claimed:

1. A process for the oxidation of cyclohexane comprising the following steps:
a) providing a reaction zone comprising a plurality of oxygen clean up zones and a plurality of oxidation zones, wherein the oxygen clean up zones and the oxidation zones are in fluid communication, and wherein the reaction zone is configured so that the oxygen clean up zones and oxidations zones are contained in a single continuous vapor space;
b) introducing a first stream comprising liquid cyclohexane into the oxygen cleanup zones;
c) introducing an oxygen containing gas into the oxidation zones;
d) passing the first stream downwardly from the oxygen clean up zones to the oxidation zones, while cross-currently passing the oxygen containing gas upwardly from the oxidation zones to the oxygen clean up zones, wherein the oxygen containing gas is introduced directly into the first stream as it travels through the oxidation zones, and wherein the reaction between the first stream and the oxygen containing gas produces a product mixture; and
e) withdrawing a product mixture from the oxidation zones that comprises cyclohexyihydroperoxide (CHHP), cyclohexanone and cyclohexanol.

2. The process of claim 1 wherein the oxygen containing gas is introduced directly into the first stream at multiple oxidation zones.

3. The process of claim 2 wherein the oxygen containing gas is introduced uniformly over the cross section of each oxidation zone.

4. The process of claim 2 wherein the oxygen containing gas is introduced directly into the first stream at each individual oxidation zone through a gas conduit.

5. The process of claim 4 wherein the gas velocity of the oxygen containing gas exiting the gas conduit is in the range from about 5 m/s to about 50 m/s.

6. The process of claim 4 wherein the oxygen containing gas exits the gas conduit at an angle of about 10 to about 50 degrees from the vertical.

7. The process of claim 4 wherein the gas conduit comprises a gas sparger.

8. The process of claim 4 wherein the gas conduit comprises a pipe with a plurality of perforations.

9. The process of claim 8 wherein the perforations have a diameter in the range of about 2 mm to about 4 mm.

10. The process of claim 8 wherein the perforations point downward at an angle of about 10 degrees to about 50 degrees from the vertical.

11. The process of claim 8 wherein a portion of the perforations point downward at an angle to the right of the vertical and a portion of the perforations point downward at an angle to the left of the vertical.

12. The process of claim 8 wherein the gas velocity of the oxygen containing gas exiting the perforations is in the range from about 5 m/s to about 50 m/s.

13. The process of claim 1 wherein the oxidation zones are maintained at a temperature range of about 145° C. to about 170° C.

14. The process of claim 1 wherein the reaction between the first stream and the oxygen containing gas takes place in the presence of a cyclohexane oxidation catalyst.

15. The process of claim 14 wherein the cyclohexane catalyst comprises soluble salts of at least one metal selected from the group comprising of cobalt and chromium.

16. The process of claim 14 wherein the cyclohexane catalyst is a soluble cobalt salt selected from a group comprising cobalt naphthenate, cobalt octoate, cobalt laurate, cobalt palminate, cobalt stearate, cobalt linoleate, cobalt acetylacetonate and mixtures thereof.

17. The process of claim 1 wherein the reaction zone comprises a single reaction vessel.
